(19) United States
(12) Patent Application Publication
(10) Pub. No.: US 2026/0061000 A1
(43) Pub. Date: Mar. 5, 2026

(54) COMPOSITIONS AND METHODS FOR THE TREATMENT OF CARDIAC ARRHYTHMIAS, INCLUDING ATRIAL FIBRILLATION

(71) Applicant: Lankenau Institute for Medical Research, Wynnewood, PA (US)

(72) Inventors: Alexander Burashnikov, Springfield, PA (US); Charles Antzelevitch, Norton, MA (US)

(73) Assignee: Lankenau Institute for Medical Research, Wynnewood, PA (US)

(21) Appl. No.: 19/104,216

(22) PCT Filed: Aug. 16, 2023
(86) PCT No.: PCT/US2023/072319

§ 371 Date: Feb. 15, 2025

Related U.S. Application Data
(60) Provisional application No. 63/371,601, filed on Aug. 16, 2022.

(51) Int. Cl. A61K 33/14; A61K 9/00; A61K 31/138; A61K 31/4458; A61P 9/06

(52) U.S. Cl. CPC A61K 33/14; A61K 9/0053; A61K 31/138; A61K 31/4458; A61P 9/06

(57) ABSTRACT

Compositions and methods for safe and effective treatment for cardiac arrhythmias via increasing [K+]0 and administering a sodium channel blocker are disclosed. In certain embodiments, the method includes administering a composition comprising sodium tablets and flecainide to a subject.

### CROSS-REFERENCE TO RELATED APPLICATION

This application claims priority of U.S. Provisional application No. 63/371,601 filed Aug. 16, 2022, the entire contents being incorporated herein by reference as though set forth in full.

### BACKGROUND OF THE INVENTION

Atrial fibrillation (AF) is the most commonly encountered arrhythmia, affecting over 3 million patients in the United States. Cardioversion of AF to normal sinus rhythm is a common clinical necessity. There is a need for improved approaches to safe and effective acute termination of AF in order to prevent its adverse effects including stroke and worsening of heart failure. An improvement in the efficacy, timing, and safety of anti-AF agents to acutely cardiovert AF is highly desirable.

It is well established that elevation of extracellular potassium ([K+]₀) depolarizes the cardiac resting membrane potential (RMP)^{1,2} and that depolarization of the RMP augments the ability of sodium channel blockers to inhibit sodium channel current (INa).³

### SUMMARY OF THE INVENTION

The present invention comprises methods for treating cardiac arrhythmia, the methods comprising, administering a sodium channel blocker to a patient having elevated extracellular potassium levels. In certain embodiments, the method for treating cardiac arrhythmia comprises administering an agent that elevates extracellular potassium levels and administering a sodium channel blocker. In certain embodiments, the agent that elevates extracellular potassium levels is administered prior to administering the sodium channel blocker. In certain embodiments, the agent that elevates extracellular potassium levels is administered at substantially the same time as the sodium channel blocker. In certain embodiments the agent is an oral potassium supplement or intravenous potassium chloride. In certain embodiments, the agent that elevates extracellular potassium levels and/or the sodium channel blocker is in a solid or liquid formulation. In certain embodiments, the extracellular potassium levels are elevated prior to administration of the sodium channel blocker.

In certain embodiments, termination of AF is increased by at least 20%, at least 30%, at least 40%, at least 50%, at least 60%, at least 70%, at least 80%, at least 90%, or at least 100%, when compared to a patient with an extracellular potassium level of 4 mM or less and/or duration of AF is decreased by at least 20%, at least 30%, at least 40%, at least 50%, at least 60%, at least 70%, at least 80%, at least 90%, or at least 100% when compared to a patient with an extracellular potassium level of 4 mM or less. In certain embodiments, the method prolongs AF cycle length when compared to a patient with baseline potassium levels. In another embodiment, the risk of ventricular proarrhythmia is decreased. In certain embodiments, the cardiac arrhythmia is atrial fibrillation and/or persistent or sustained AF.

In certain embodiments, the cardiac arrythmia is resistant to treatment with a sodium channel blocker alone. In another embodiment, the elevated extracellular potassium level is greater than 4 mM, greater than or equal to 5 mM, between 5 mM and 5.5 mM, about 5.5 mM, or about 4 mM. In certain embodiments the baseline potassium level is between 2.0 and 4.5 mM, 2.5 and 4.5 mM, or 3.0 and 4.0 mM. In certain embodiments the elevated potassium level is at least 0.5 mM, 0.5-0.8 mM, or 1.0 mM greater than the baseline potassium levels. In certain embodiments, the sodium channel blocker is a class IC sodium channel blocker and may be selected from Flecainide or Propafenone. In certain embodiments the sodium channel blocker is a class IB or IA sodium channel blocker. In certain embodiments, the sodium blocker is administered by oral inhalation.

Another aspect of the invention comprises a method of treating cardiac arrhythmia, the method comprising administration of an agent that elevates extracellular potassium levels above baseline levels. In certain embodiments, the elevated extracellular potassium levels are about 5.5 mM. Yet another aspect of the invention comprises a method of treating cardiac arrhythmia, the method comprising administration of an agent that elevates extracellular potassium levels by at least 0.5 mM. In certain embodiments, extracellular potassium levels are elevated by 0.5-0.8 mM. In certain embodiments, extracellular potassium levels are elevated by 1.0 mM.

Another aspect of the invention comprises a kit for performing any of the preceding methods, the kit comprising a sodium channel blocker and an agent that elevates extracellular potassium levels. Another aspect of the invention comprises an oral dosage composition comprising a sodium channel blocker and an agent that lowers extracellular potassium levels. In certain embodiments, the sodium channel blocker is Flecainide or Propafenone.

Still other aspects and advantages of these compositions and methods for making the compositions and using the compositions are described further in the following detailed description of the preferred embodiments thereof.

### BRIEF DESCRIPTION OF THE FIGURES

FIG. 1. Unfolded isolated canine arterially-perfused right atrial preparation. Atrio-ventricular ring (AVR), pectinate muscle (PM), crista terminalis (CT), the right atrial appendage (APG), inferior and superior vena cava (IVC and SVC).

FIG. 2. Effect of extracellular potassium concentration in altering the efficacy of flecainide to cardiovert atrial fibrillation (AF). Numbers above the bars indicate the proportion of atrial preparations successfully cardioverted.

FIG. 3. Effect of extracellular potassium concentration on the timing of flecainide-induced cardioversion of atrial fibrillation (AF). *P<0.05 vs “4 mM [K⁺]₀+Flecainide.” n=3-5, reflecting the number of preparations in which AF was cardioverted (see FIG. 2).

FIG. 4. Examples of termination of atrial fibrillation by a combination of flecainide and mild elevation of [K]₀ (top) or elevated [K⁺]₀ alone (bottom). Pseudo-electrocardiogramacordings are shown. ACh=acetylcholine.

FIG. 5. Cardioversion of AF by flecainide and mild elevation of [K⁺]₀ is associated with a rate-dependent depression of atrial excitability, causing loss of 1:1 activation. The data were obtained immediately following AF cardioversion by flecainide (1.5 μM) and 6 mM [K⁺]₀. The gradual reduction of action potential (AP) and ECG amplitudes following acceleration of pacing CL from 500 to 150 ms indicate a cumulative inhibition of the sodium channel, resulting in failure of 1 to 1 activation.

FIG. 6. Effect of a mild elevation of [K⁺]₀ with and without flecainide on action potential waveform in atria and ventricles. CL=500 ms.

FIG. 7. Atrial-selective effects of a mild elevation of [K⁺]₀ and flecainide (1.5 μM) separately and in a combination on effective refractory period (ERP) and post-repolarization refractoriness (PRR), surrogates for ability of drugs to suppress AF. Pacing CL=500 ms. *p<0.05 vs. respective control. **p<0.05 vs. respective [K+]0=6 mM. #0.001 vs. respective control. n=5 for each datapoint in experiments reporting the effects of [K⁺]₀ elevation (from 4 to 6 mM) with and without flecainide. n=6 for each datapoint in the experiments reporting the effect of flecainide at [K⁺]₀=4 mM. APD-action potential duration.

FIG. 8. Atrial selective depression of excitability by flecainide alone and a combination with mild elevation of [K⁺]₀ (6 mM). *p<0.05 vs. respective control. **p<0.05 vs. respective [K⁺]₀=6 mM. DTE-diastolic threshold of excitation. Pacing CL=500 ms. n=5-6 (see FIG. 7)

FIG. 9. Schematic illustrating the rationale for the “Elevated of [K⁺]₀+I_{Na} block for AF cardioversion” approach. Elevation of [K⁺]₀ depolarizes the atrial resting membrane potential (RMP) resulting in inactivation of the sodium channels. The reduced level of I_{Na} manifests as depressed atrial excitability and prolongation of atrial refractoriness, leading to more effective termination of AF.

### DETAILED DESCRIPTION OF THE INVENTION

Cardioversion of atrial fibrillation (AF) is a common clinical necessity and there is a need for more effective and safe options for acute cardioversion of AF. It is shown herein that the efficacy and timing of AF cardioversion by sodium channel current (I_{Na}) block can be improved by mild elevation of extracellular potassium ([K⁺]₀). Using a canine acetylcholine-mediated AF model (isolated coronary-perfused right atrial preparations with a rim of right ventricle), the ability of flecainide to suppress AF in the presence of [K⁺]₀ ranging from 3 to 8 mM was evaluated.

At [K⁺]₀ of 4 mM, (baseline) sustained AF (>1 hour) was induced in 5/5 atria in the presence of 0.5 μM acetylcholine. Flecainide (1.5 M) cardioverted 3/6 atria at 4 mM [K⁺]₀, 1/6 atria at 3 mM [K⁺]₀, 5/5 atria at 5 and 6 mM [K⁺]₀, and 4/4 atria at 8 mM [K⁺]₀. In the absence of flecainide, an increase in [K⁺]₀ from 4 (control) to 5, 6 and 8 mM terminated AF in 0/5, 2/6, and 4/4 atria. The time to conversion was abbreviated by elevation of [K⁺]₀ from 4 to 8 mM. AF cardioversion was associated with rate-dependent depression of excitability. Following AF termination with flecainide and elevated [K⁺]₀, AF was either not inducible or induced for a very brief episode (<100 sec). Combined flecainide and elevated [K⁺]₀ (6 mM) caused an atrial preferential depression of excitability.

These findings indicate that a combination of an I_{Na} blocker accompanied by mild elevation of plasma potassium is a novel approach to more effectively, rapidly, and safely cardiovert AF and prevent its recurrence.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

It is also contemplated that the term “compound” or “compounds” refers to the compounds discussed herein and includes precursors and derivatives of the compounds, including acyl-protected derivatives, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

As used herein, the term “prodrug” refers to a protected form of the compound, which release the compound after administration to a subject. For example, a compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the present disclosure. Thus, the term “prodrug” refers to a metabolic precursor of a compound of the present disclosure that is pharmaceutically acceptable. A prodrug may be inactive when administered to a subject in need thereof, but may be converted in vivo to an active compound of the present disclosure. Prodrugs are typically rapidly transformed in vivo to yield the parent compound of the present disclosure, for example, by hydrolysis in blood. The prodrug compound often offers advantages of solubility, tissue compatibility or delayed release in a subject.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, amelioration, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

The phrase “cardiac arrhythmia” refers to the improper beating of the heart, whether irregular, too fast, or too slow. Cardiac arrhythmia occurs when electrical impulses in the heart don't work properly. In certain patients, there may be no symptoms. In other patients, symptoms may include a fluttering in the chest, chest pain, fainting, or dizziness.

The phrase “supraventricular tachycardia” refers to a faster than normal heart rate arising from activity above the heart's two lower chambers. Supraventricular tachycardia is a rapid heartbeat that develops when the normal electrical impulses of the heart are disrupted. In certain embodiments, the tachycardia refers to heartbeats of at least 100 bpm. In preferred embodiments, the patient has heart beats at about 150-220 bpm or faster. Certain patients may exhibit symptoms like heart palpitations, fluttering or pounding in the chest, pounding sensation in the neck, weakness or feeling very tired (fatigue), chest pain, shortness of breath, lightheadedness or dizziness, sweating, and fainting (syncope) or near fainting. In some patients, there may be no symptoms at all.

The phrase “atrial fibrillation”, “A-fib”, or “AF” refers to an irregular and often very rapid heart rhythm (arrhythmia) that can lead to blood clots in the heart. During A-fib, the atria beat chaotically, irregularly, and out of sync with the ventricles. AF may cause a fast, pounding heartbeat, palpitations, shortness of breath and/or weakness. Patient with AF haven an increased risk of stroke, heart failure and other heart-related complications. Episodes of atrial fibrillation may come and go, or they may be persistent. Persistent AF refers to patients that have an AF episode lasting more than 7 days. Persistent AF is resistant to currently available pharmacological therapies. I_{Na} blockers, generally effective for cardioversion of paroxysmal AF (i.e., AF lasting up to 6 days), lose their anti-AF efficacy in patients with persistent AF. A major reason for this failure is that in patients with persistent AF atrial cells display a more negative or hyperpolarized resting membrane potential which reduces the ability of I_{Na} blockers to depress excitability, thus rendering these drugs less effective for termination of AF. Elevation of [K⁺]₀ shifts the resting membrane potential of atrial cells in a positive direction, thus enhancing the ability of I_{Na} blockers to inhibit I_{Na} and depress excitability. As a consequence, elevation of plasma [K⁺]₀ restores or improves the effectiveness of I_{Na} blockers to cardiovert persistent AF.

The phrase “sodium channel” refers to transmembrane proteins that underlie rapid depolarization due to entry of sodium into the cells thus initiating action potentials in electrically excitable cells, such as neurons and cardiac myocytes. The phrase “sodium channel current” or “I_{NA}” refers to the movement of sodium ions through the channel across a membrane.

“Sodium channel blockers” are a group of drugs which impair the conduction of sodium ions through sodium channels. Sodium channel blockers can be administered orally, intravenously, or by oral inhalation. The phrase “Class I” or “Class I sodium channel blockers” refers to sodium channel blockers that are used in the treatment of cardiac arrhythmia. Type I agents are grouped by their effect on the Na+channel, and by their effect on cardiac action potentials. Class IA sodium channel blockers block the fast sodium channel, which depresses the phase 0 depolarization (i.e., reduces Vmax), and slows conduction. Class IA sodium channel blockers also inhibit potassium channel, which prolongs the action potential duration. Agents in this class cause decreased conductivity and increased refractoriness. Indications for Class IA agents are supraventricular tachycardia, atrial fibrillation, ventricular tachycardia, symptomatic ventricular premature beats, and prevention of ventricular fibrillation. Class IA agents include quinidine, procainamide, and disopyramide.

Class IB sodium channel blockers either do not change the action potential duration or shorten the action potential duration, but commonly prolong refractoriness in the atrium due to induction of post-repolarization refractoriness. These agents commonly decrease Vmax with fast response action potentials. Class IB agents are indicated for the treatment of ventricular tachycardia and symptomatic premature ventricular beats, and prevention of ventricular fibrillation. Class IB agents include lidocaine, mexiletine, tocainide, and phenytoin.

Class IC sodium channel blockers markedly depress the phase 0 depolarization (decreasing Vmax). They decrease conductivity, but have a minimal effect on the action potential duration. Of the sodium channel blocking antiarrhythmic agents (the class I antiarrhythmic agents), the class IC agents have the most potent sodium channel blocking effects. Class IC agents are indicated for supraventricular arrhythmias such as atrial fibrillation. Class IC agents include encainide, flecainide, moricizine, and propafenone.

A general limitation for the use of Class IC I_{Na} blockers (including flecainide) in patients with AF is the risk of induction of ventricular pro-arrhythmia in patients with significant structural heart diseases. However, when combined with elevated [K⁺]ₒ lower dosages of Class IC I_{Na} blockers can be used, thus minimizing the risk of ventricular proarrhythmia. Elevating [K⁺]ₒ. in combination with IC agents can therefore improve safety of the sodium channel blockers.

The phrase “extracellular potassium concentration” or [K⁺]₀ refers to the concentration of potassium in the extracellular space. More specifically, extracellular potassium refers to the concentration of potassium in the extracellular space of cardiac cells. Under baseline conditions or normokalemic potassium conditions, extracellular potassium concentration is approximately 4 mM. In certain embodiments, the baseline potassium level is between 2.5-5.0 mM. In certain embodiments, a mild elevation of [K⁺]₀ to up to 5 mM is within normal physiological range. Hypokalemic conditions refers to a [K⁺]₀ of less than 3.5 mM. Hyperkalemic conditions refer to a [K⁺]₀ of at least 5.0 mM. In certain embodiments, hyperkalemic conditions refer to a [K⁺]₀ of at least 5.2 mM. In certain embodiments, hyperkalemic conditions refer to a [K⁺]₀ of at least 5.3 mM. In certain embodiments, hyperkalemic conditions refer to a [K⁺]₀ of at least 5.4 mM. In certain embodiments, hyperkalemic conditions refer to a [K⁺]₀ of at least 5.5 mM. Elevated levels of [K⁺]₀ refers to patients with [K⁺]₀ of at least 4 mM, provided that the baseline plasma potassium is >3.5 mM In certain embodiments, elevated potassium levels are greater than or equal to 5 mM. In certain embodiments, the elevated potassium levels are less than or equal to 5.5 mM. In certain patients, baseline serum levels are significantly lower than 4 mM. In certain embodiments, these patients have baseline serum levels of potassium of lower than 3 or 3.5 mM. In these patients, elevated levels of [K⁺]₀ are greater than or equal 4 mM. In certain embodiments, the elevated levels of [K⁺]₀ in these patients is between 4 mM and 5.5 mM. In certain embodiments elevated levels of [K⁺]₀ is a extracellular potassium level at least 0.5 mM greater than the patient's baseline [K⁺]₀₊. In certain embodiments elevated levels of [K⁺]₀ is an extracellular potassium level between 0.5 mM-0.8 mM greater than the patient's baseline [K⁺]₀. In certain embodiments elevated levels of [K⁺]₀ is an extracellular potassium level at least 1 mM greater than the patient's baseline [K⁺]₀₊. In certain embodiments, elevated potassium levels do not exceed about 5.5 mM. In certain embodiments, elevated potassium levels do not exceed about 5.6 mM. In certain embodiments, elevated potassium levels do not exceed about 5.7 mM. In certain embodiments, elevated potassium levels do not exceed about 5.8 mM. In certain embodiments, elevated potassium levels do not exceed about 5.9 mM. In certain embodiments, elevated potassium levels do not exceed about 6.0 mM.

Various compositions are known by those skilled in the art to increase [K⁺]₀ levels. In one embodiment, [K⁺]₀ levels are elevated by administration of oral potassium tablets or intravenous potassium chloride.

The phrase “cardiac action potential” or “AP” refers to a brief change in voltage (membrane potential) across the cell membrane of heart cells caused by the movement of charged atoms (called ions) between the inside and outside of the cell, through proteins called ion channels. The cardiac action potential differs from action potentials found in other types of electrically excitable cells, such as nerves. Action potentials also vary within the heart; this is due to the presence of different ion channels in cells in different regions of the heart. The cardiac action potential stimulates muscle cells of the atrial myocardium to depolarize and contract in unison in systolic contraction, after which the cardiac action potential encounters the atrioventricular node located at the juncture of the atria and ventricles near the center of the heart. The atrioventricular node slightly delays cardiac action potential propagation to ensure complete drainage of blood from the atria after which the muscle cells of the ventricular myocardium are stimulated into systolic contraction and thereby complete the heartbeat cycle. The electrical activation of the heart can be measured using an ECG.

### Methods of Treatment

Provided herein are methods of treatment of cardiac arrhythmias including supraventricalular tachycardia, atrial tachycardia, and atrial fibrillation. In certain embodiments, the AF is persistent or sustained. The methods include administration of an effective amount of at least one sodium channel blocker together with at least of one means of elevating plasma potassium (potassium supplement [oral solid or liquid formulations] or IV). In certain embodiments, the sodium channel blocker is a class IC blocker. In certain embodiments, the sodium channel blocker is selected from encainide, flecainide, moricizine, and propafenone. In some embodiments, the symptoms of the cardiac arrhythmia are reduced, as compared to a control. In certain embodiments, the sodium channel blocker and the means of elevating plasma potassium are administered sequentially. By sequential administration, the sodium channel blocker may be delivered to a subject before or after administration of the means of elevating plasma potassium. In certain embodiments, the sodium channel blocker may be delivered to a subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes before or after administration of the means of elevating plasma potassium. In further embodiments, the sodium channel blocker may be delivered to a subject at least 1, 2, 3, 4, 5, 10, 15, 16, 17, 18, 19, 20, 21, 22, 23, or 24 hours before or after administration of the means of elevating plasma potassium. In further embodiments, the sodium channel blocker may be delivered to a subject at least 1, 2, 3, 4, 5, 6, 7, 8, 9, 10, 11, 12, 13, or 14 days, or 1 week, 2 weeks, 3 weeks, or 4 weeks before or after administration of the means of elevating plasma potassium. In still further embodiments, the sodium channel blocker may be delivered to a subject in any combination of months, days, hours, minutes, and seconds within these ranges. In further embodiments, the administration of the sodium channel blocker and means of elevating plasma potassium may be repeated multiple times.

The term “increases” or “elevates” means to raise the extracellular concentration of a specific component, e.g., potassium. Extracellular Potassium concentrations can be compared to a control or to a standard level, or the subject's level prior to administration of the agent. An increase can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more higher than the standard.

In certain embodiments, the subject has an elevated [K⁺]₀ after administration of an effective amount of agent that elevates extracellular potassium concentration. In certain embodiments the agent is an oral potassium tablet(s), or intravenous potassium chloride.

In certain embodiments, the agent elevates the extracellular potassium concentration to at least 4 mM, at least 5 mM, at least 5.1 mM, at least 5.2 mM, at least 5.3 mM, at least 5.4 mM, or about 5.5 mM.

In certain embodiments, the method of treatment effectively suppresses symptoms associated with cardiac arrhythmia. In some embodiments, symptoms of cardiac arrhythmia include a fluttering in the chest, chest pain, fainting, and/or dizziness.

In certain embodiments, the method of treatment effectively suppresses symptoms associated with supraventricular tachycardia. In some embodiments, symptoms of supraventricular tachycardia include heart palpitations, fluttering or pounding in the chest, pounding sensation in the neck, weakness or feeling very tired (fatigue), chest pain, shortness of breath, lightheadedness or dizziness, sweating, and/or fainting (syncope) or near fainting.

In certain embodiments, the method of treatment effectively suppresses symptoms associated with atrial fibrillation. In some embodiments, symptoms of atrial fibrillation include a fast, pounding heartbeat, palpitations, shortness of breath and/or weakness. In certain embodiments, the treatment reduces the risk of stroke, heart failure, and other heart-related complications.

### Administration

The agents used in the methods described herein can be formulated for enteral, parenteral, topical, or cardiac administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

In certain embodiment, the agents administered in the methods described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with cardiac arrhythmias, supraventuicular tachycardia, and/or atrial fibrillation.

The exact amount of the described agents required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The compositions are administered in an effective amount and for a period of time effect to reduce one or more symptoms associated with the disease to be treated. It should be understood that the “effective amount” for a composition which comprises a sodium channel blocker, an agent that increases extracellular potassium, or a modification thereof, may vary. In one embodiment an effective amount includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body weight. In another embodiment, the range of effective amount is 40 to 50 mg/kg body weight. In another embodiment, the range of effective amount is 1 to 50 mg/kg body weight. Still other doses falling within these ranges are expected to be useful.

In another embodiment, the range of effective drug concentrations is 0.001 mg to 10 g. In another embodiment, the range of effective drug concentrations is 0.01 mg to 1 g. In another embodiment, the range of effective amount is 0.01 mg to 100 mg. In another embodiment, the range of effective amount is 0.1 mg to 100 mg. In another embodiment, the range of effective amount is 0.1 mg to 500 mg. In another embodiment, the range of effective amount is 1 mg to 100 mg. In another embodiment, the range of effective amount is 10 mg to 500 mg. In another embodiment, the range of effective amount is 10 mg to 750 mg. In another embodiment, the range of effective amount is 0.01 mg to 100 mg. In another embodiment, the range of effective amount is 1 mg to 500 mg. In another embodiment, the effective amount is 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, or 1000 mg.

In certain embodiments, a dosage of potassium chloride of up to about 10mEq/hr IV to a maximum amount of about 200 mEq/day is provided. In other embodiments, a dosage of potassium chloride of up to about 40 mEq/hr IV to a maximum amount of about 400 mEq/day is provided.

In certain embodiments, the dosage of the sodium channel blocker is at least 100-900 mg/day. In other embodiments, the dosage of the sodium channel blocker is about 100-200 mg/day. In other embodiments the dosage of the sodium channel blocker is at least 100 mg/day. In other embodiments, the dosage of the sodium channel blocker is about 450-900 mg/day. In certain embodiments, the maximum amount of the sodium channel blocker does not exceed 900 mg/day. In certain embodiments, the maximum amount of the sodium channel blocker does not exceed 400 mg/day. In other embodiments, the maximum amount of the sodium channel blocker does not exceed 300 mg/day.

### Kits and Articles of Manufacture

Any of the aforementioned products can be incorporated into a kit which may contain an agent that increase extracellular potassium concentration and a sodium channel blocker, a pharmaceutically acceptable carrier, instructions for use, a container, a vessel for administration, or any combination thereof.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Materials And Methods

This investigation conforms with the Guide for Care and Use of Laboratory Animals. Hearts from purpose-bred adult male Beagle dogs (10-16 kg, 10-19 months old) were obtained from Envigo (Denver, PA). Dogs were sedated with ketamine (10 mg/kg, intramuscularly [IM]) and xylazine (2 mg/kg, IM). Prior to euthanasia with Euthasol solution (pentobarbital sodium and phenytoin sodium; 0.22 ml/kg, intravenously [IV]), heparin (human pharmaceutical grade, 1,000 U/kg, IV) was administered. The heart was then isolated via a left thoracotomy. Retrograde aortic perfusion of the hearts with ice-cold cardioplegic solution (modified Krebs-Henseleit solution [K-H]; 16 mM KCl) was immediately performed to clear the coronary vasculature of blood. The hearts were then placed in a sealed container containing ice-cold cardioplegic solution and transported to our institution via a private courier in an insulated package surrounded by icepacks.

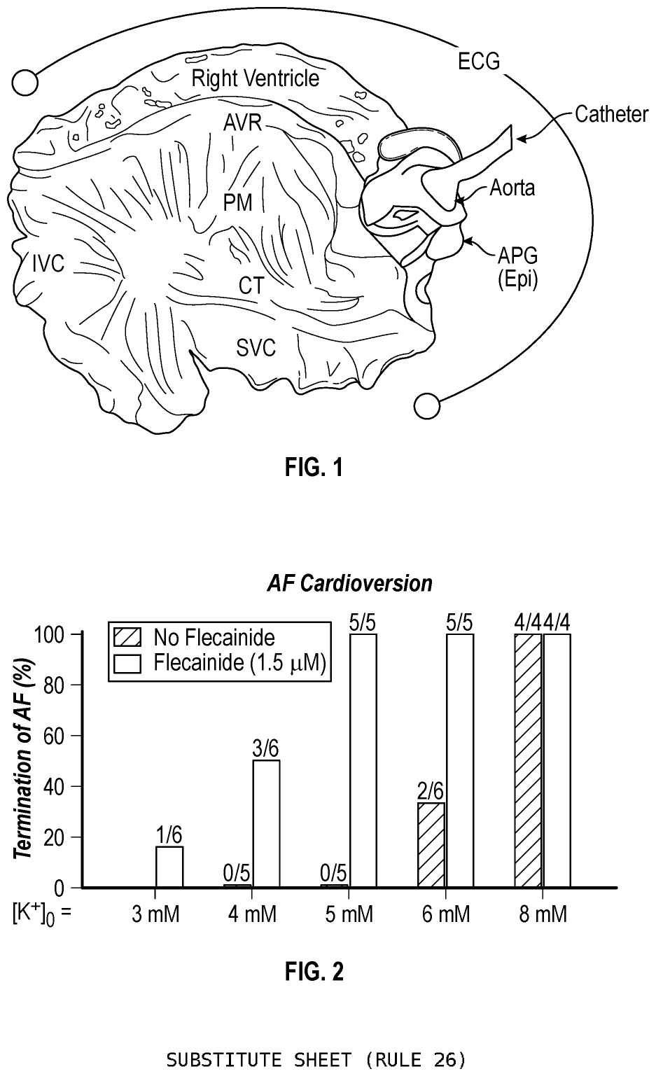

Experiments were performed using isolated arterially-perfused canine preparations perfused with Tyrode's solution containing (in mM): NaCl 129, KCl 4, NaH₂PO₄ 0.9, NaHCO₃ 20, CaCl₂ 1.8, MgSO₄ 0.5, glucose 5.5, insulin lunit/liter, bubelled with 95% O2 and 5% CO₂. pH=7.4. Preparations consisting of the right atrium with a rim of right ventricle (1.0-2.0 cm) were isolated from the canine hearts (FIG. 1). The ostium of the right coronary artery was cannulated with polyethylene tubing, and the preparations were perfused with cold Tyrode's solution (8°-12° C.) containing 8.5 [K⁺]₀. With continuous coronary perfusion, all ventricular branches of the right coronary artery were immediately clamped with metal clips (1.5×10 mm; Kent Scientific Corp., Torrington, CT). The total time from excision of the heart to cannulation and perfusion of the artery was 4 minutes. Ventricular and atrial coronary branches were ligated using silk thread. The preparations were placed in a temperature-controlled bath (8×6×4 cm) and perfused at a rate of 10-15 mL/min using Tyrode's solution. The temperature was maintained at 37° C.±0.5° C. (PolyStat; Cole Parmer Instrument, IL). The perfusate was delivered to the artery by a roller pump (MasterFlex L/S, Cole Parmer Instrument). An air trap was used to avoid bubbles in the perfusion line.

Transmembrane action potential (AP) recordings (sampling rate 41 kHz) were obtained from the right atrium and ventricle using floating glass microelectrodes (10-25 MΩ DC resistance). Glass microelectrodes were connected to a high-input impedance intracellular amplifier (Electro-705; World Precision Instruments, Sarasota, FL). A pseudo-electrocardiogram (ECG) was recorded using two electrodes consisting of Ag/AgCl half cells placed in the Tyrode's solution bathing the preparation, 1.0 to 1.2 cm from opposite ends of the atrial coronary-perfused preparations (FIG. 1). The signals were amplified, digitized, and analyzed (Cambridge Electronic Design, Cambridge, England). Effective refractory period (ERP) was measured by delivering premature stimuli after every 10th basic beat at a pacing cycle length (CL) of 500 ms. The diastolic threshold of excitation (DTE) was determined by increasing stimulus intensity in 0.01 mA steps starting from 0.1 mA, until a steady 1:1 activation was achieved. Post-repolarization refractoriness (PRR) was recognized when ERP exceeded an action potential duration at 90% repolarization (APD₉₀) in the ventricle and action potential duration at 70% repolarization (APD₇₀) in atria. Ventricular ERP coincides with APD₉₀, whereas atrial ERP generally coincides with APD₇₀.⁴ The electrophysiological measurements were performed following at least 20 min of perfusion with flecainide and 5 min with elevated potassium concentrations. The electrophysiological measurements were obtained at a CL of 500 ms.

A well-established acetylcholine (ACh)-mediated non-self-terminating (persistent) AF experimental model was used in the current study.⁴ AF was induced by either delivering a single premature stimulus or by rapid pacing (CLs=40-80 ms, for 2-5 seconds). The efficacy and time course for flecainide to cardiovert AF (induced at 4 mM potassium) was studied at 4 mM (baseline) and at 5, 6, and 8 mM of potassium concentration ([K⁺]₀). Sustained AF (here defined as lasting >1 hour) was induced in 100% experiments in the presence of 4 mM [K⁺]. 5-7 minutes after non-self-terminating AF was first introduced at 4 mM [K⁺]₀, flecainide was added to the Tyrode's perfusate containing either 4 mM [K⁺]₀ or with a concomitant increase in potassium concentration (to 5, 6, or 8 mM).

Additionally, the capability of elevated potassium (5, 6, and 8 mM) alone to terminate AF was investigated. In these experiments, sustained AF was first induced at 4 mM [K⁺]₀ and 5-7 minutes later potassium concentrations were increased (to 5, 6, or 8 mM). To mimic hypokalemia, AF termination efficacy of flecainide was tested at 3 mM [K⁺]₀. If AF was not terminated by the tested conditions in 45 minutes, the AF was considered as “non-terminating”. In the experiments in which AF was cardioverted by tested conditions tested, electrical re-induction of AF was attempted within 1-5 minutes after AF termination (≥5 attempts in ≥2 atrial locations).

Overall, 41 right atrioventricular preparations isolated from 41 canine hearts were used in the study, with 46 experiments investigating AF cardioversion and 11 experiments investigating electrophysiological parameters in atria and ventricles. Thirty preparations were used either exclusively for studying termination of 1 episode of AF in each preparation (n=19) or for studying the first AF cardioversion (n=11) out of 2 tested. In these 11 preparations, the first AF episode was terminated within 10 minutes (by elevated [K⁺]₀ or elevated [K⁺]₀+flecainide). The second episode of AF cardioversion was investigated after a ≥30-minute washout period (with control solution) and full recovery of the atrial electrophysiological parameters (ERP, DTE, and APD). Five AF cardioversion experiments were performed in 5 separate preparations after washout (≥30 minutes) of elevated [K⁺]₀ (from 4 to 6 mM) and flecainide. Six atrial preparations were used for the measurement of electrophysiological parameters only. The choice of the interventions for AF cardioversion was randomly selected.

ACh and flecainide (Sigma-Aldrich, St. Louis MO) were dissolved in distilled water and prepared fresh as a 10 mM stock solution before each experiment.

Statistical analysis of the data was performed using one way repeated measures analysis of variance or Student's t-test, as appropriate. Data are reported as mean±SD.

### Example 2: Effect of [k+] Elevation on Atrial Fibrillation

### AF Cardioversion

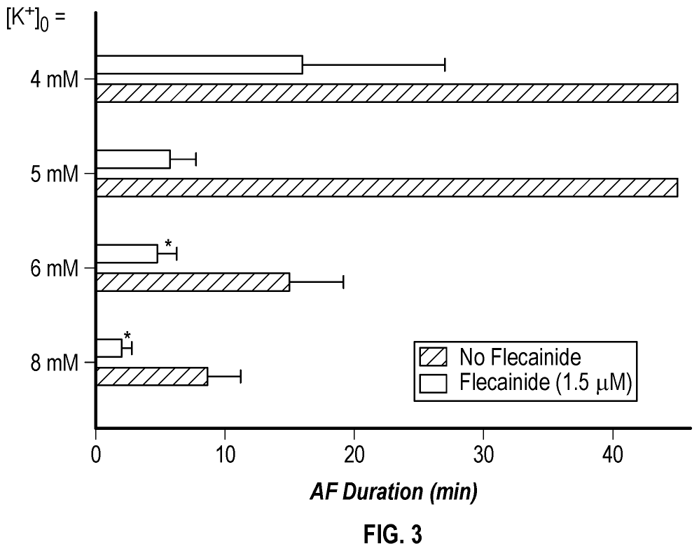

In the presence of ACh (0.5 μM), premature stimulation or rapid pacing readily induced non-self-terminating AF (>1 hour) in 5/5 atria under baseline conditions ([K⁺]₀=4 mM). The efficacy of flecainide (1.5 μM) to cardiovert ACh-mediated AF (induced at 4 mM [K⁺]₀) was moderately effective at 4 mM of [K⁺]₀ and highly effective when [K⁺]₀ was elevated during the on-going AF to 5, 6, and 8 mM of (FIG. 2). Importantly, even a mild elevation of [K⁺]₀ within normal physiological range (from 4 to 5 mM) resulted in a substantial improvement in AF cardioversion by flecainide (FIG. 2). An increase of [K⁺]₀ from 4 to 5 mM alone (without flecainide) did not cardiovert the arrhythmia (FIG. 2). Elevation of [K⁺]₀ from 4 to 6 or 8 mM during sustained AF, terminated AF in 40% and 100% of experiments, respectively (FIG. 2). The time course of flecainide-mediated AF termination was abbreviated by the elevation of [K⁺]₀ (FIG. 3).

Under simulated hypokalemic conditions ([K⁺]₀=3 mM) addition of flecainide (1.5 μM) to the coronary perfusate during on-going sustained AF cardioverted the arrhythmia in only 1 out 6 atria tested (FIG. 2).

Following AF cardioversion by flecainide under “normokalemic” potassium concentration (4 mM), AF or atrial tachycardia (AT) was readily inducible in 3/3 preparations, but these episodes self-terminated in ≤5 min. Following AF cardioversion with a combination of flecainide and elevated K⁺π₀ (5-8 mM), AF/AT were either not inducible or induced for very brief episodes (lasting <100 sec, in most cases <10 sec). In two experiments in which 6 mM [K⁺]₀ alone terminated AF, AF/AT was readily inducible in both; in one AF lasted 20 min. In the experiments with 8 mM [K⁺]₀ alone, AF was readily inducible in 4/4 atria, but in all cases the AF episodes were brief (≤26 sec). In the only atria in which flecainide terminated AF in the setting of hypokalemia (3 mM [K⁺]₀), sustained episodes of AF were readily re-induced (≤31 min duration).

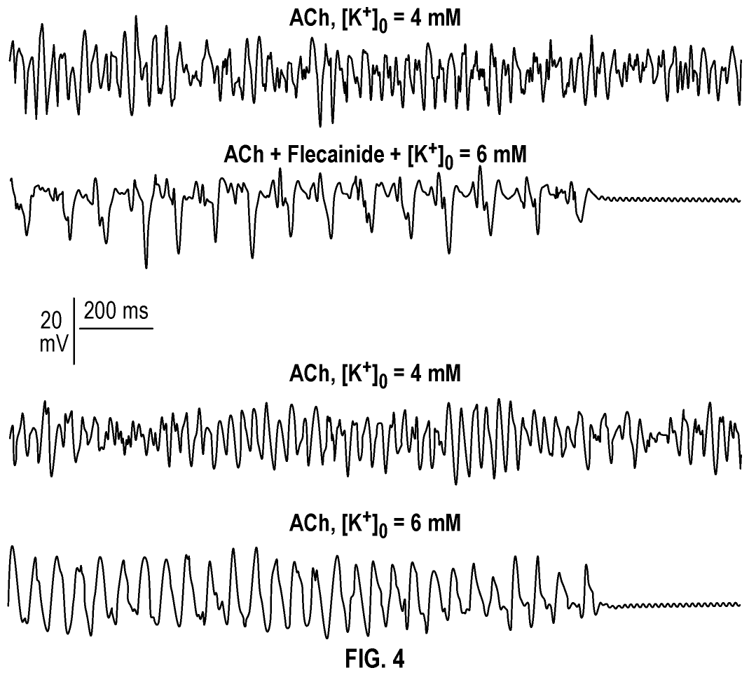

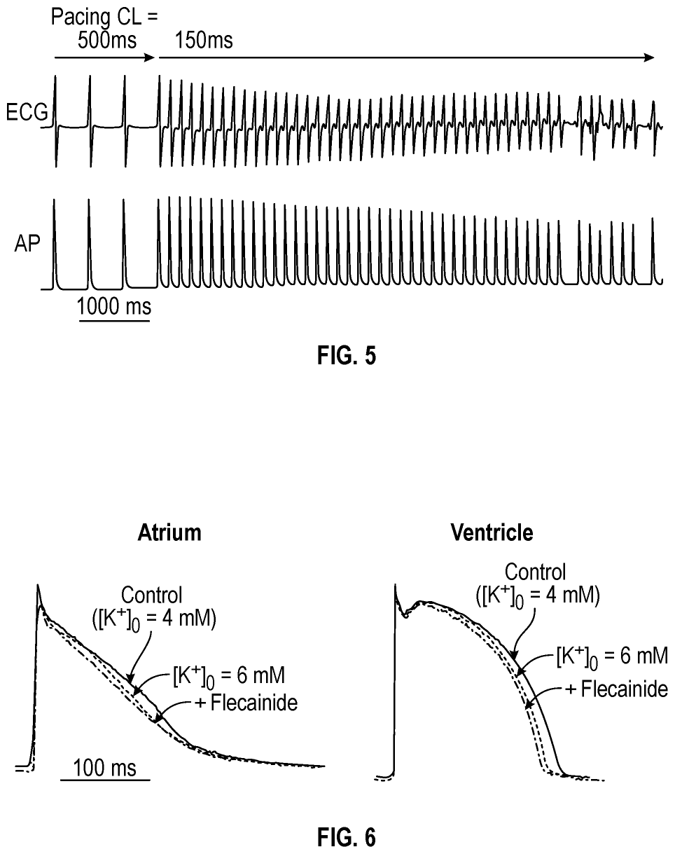

Cardioversion of AF in all tested condition was accompanied with a significant prolongation of AF cycle length (AFCL, FIG. 4). AF termination was associated with rate-dependent depression of excitability, preventing rapid activation of the atria (FIG. 5).

### Atrial Selectivity

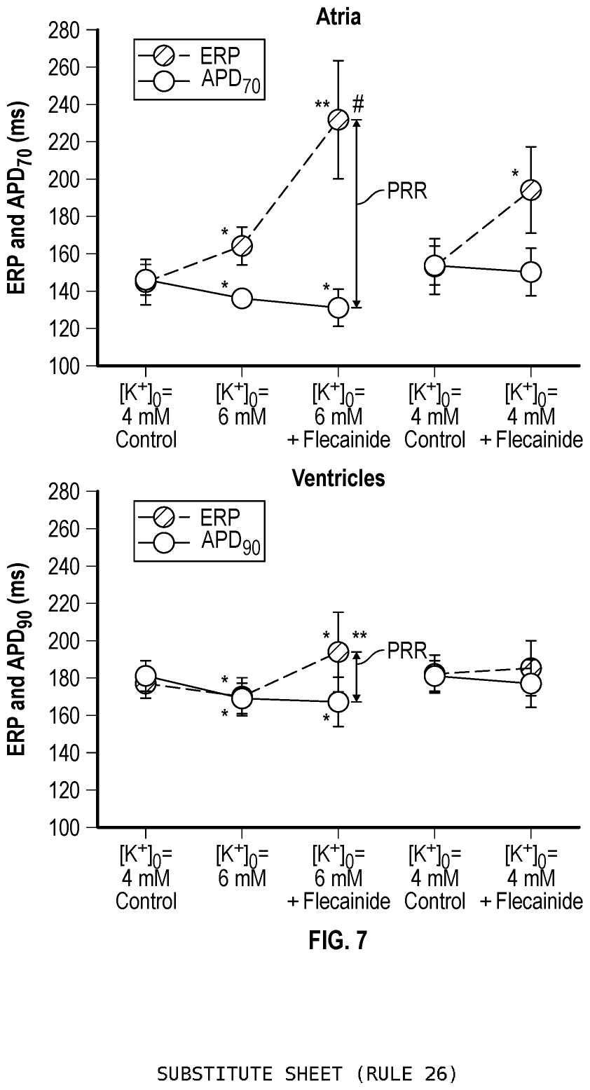

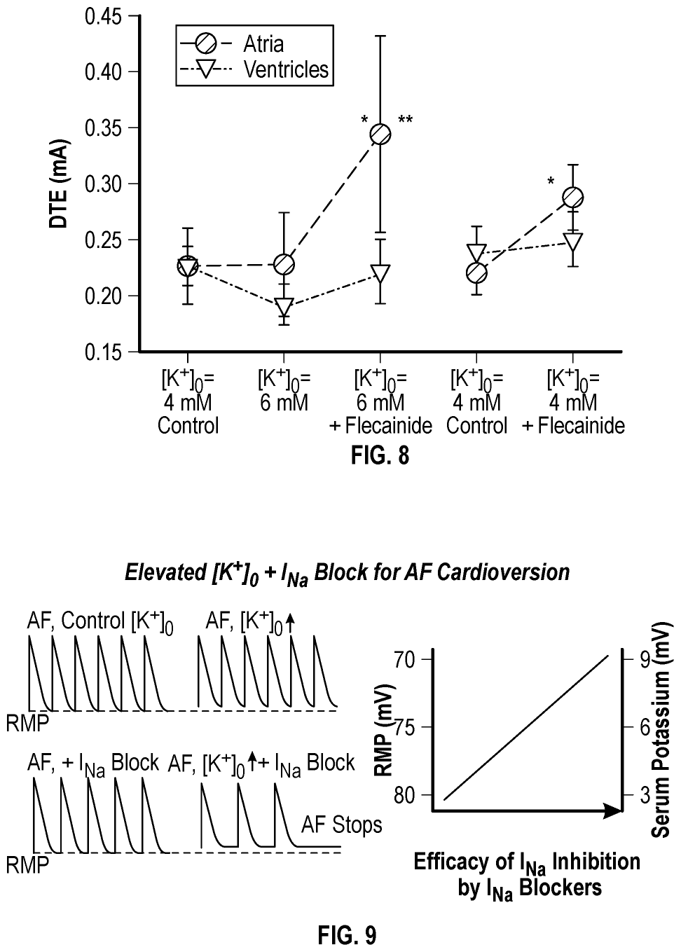

In another series of experiments, we sought to determine the atrial selectivity of the elevated [K⁺]₀ plus flecainide combination by comparing the response in the ventricle vs. atrium. Elevation of [K⁺]₀ from 4 to 6 mM significantly abbreviated both APD and ERP in the ventricles but abbreviated only APD while significantly prolonging ERP in the atria (FIGS. 6 and 7). This atrial-selective ERP prolongation was due to an atrial-selective induction of post-repolarization refractoriness (PRR) by the mild elevation of [K⁺]₀ (FIG. 7). The addition of flecainide to the coronary perfusate containing 6 mM potassium produced prolongation of ERP in both atria and ventricle but this was much more pronounced in the atria, again due to an atrial predominant development of PRR (FIG. 7). At a [K⁺]₀ of 4 mM, flecainide caused no significant change in APD in both atria and ventricles but produced a substantial lengthening of ERP in the atria (FIG. 8).

DTE was not significantly affected by 6 mM [K⁺]₀ (vs 4 mM) in either atria or ventricles, although there was a tendency of DTE decrease in the ventricles, not reaching statistical significance (FIG. 8). The addition of flecainide in the presence of 6 mM [K⁺]₀ caused a large atrial-selective increase in DTE (FIG. 8). At [K⁺]0 of 4 mM, flecainide alone produced a moderate atrial predominant increase in DTE (FIG. 8).

### DISCUSSION

The data presented herein provides experimental evidence indicating that a moderate elevation of [K⁺]₀, by as little as 1 mM significantly improves the efficacy of sodium channel blockers and abbreviates the time course for acute conversion of AF to sinus rhythm by the sodium channel blocker flecainide. A combination of flecainide and elevated [K⁺]₀ caused an atrial predominant depression of excitability, pointing to a relatively low risk of induction of ventricular pro-arrhythmia. A combination of sodium channel block with a mild elevation of extracellular potassium improves the efficacy of sodium channel block to cardiovert AF and abbreviates the time for cardioversion.

No published studies to our knowledge have demonstrated or suggested the “elevated [K⁺]₀+I_{Na} block for AF”approach for termination of AF.

The effectiveness of I_{Na} blockers for acute cardioversion of AF is well established. However, several important limitations have been identified, including the risk of induction of ventricular proarrhythmia and significant reduction of their cardioverting efficacy against persistent AF.⁵ elevation of [K⁺]₀ itself reduces the availability of the sodium channel (secondary to a depolarization of atrial RMP), 1,2 that may cardiovert AF. It has been reported that elevation of [K⁺]₀ from 4 to 6-8 mM cardioverts ACh-mediated AF in 3/4 isolated pig atria.⁶ There are also many clinical case-reports demonstrating that severe hyperkalemia (>8 mM serum [K⁺]₀) can terminate persistent/permanent AF.⁷⁻⁹ In patients with recent-onset AF, a mild elevation of plasma potassium (via KCI infusion) within the low-normal range may cardiovert the arrhythmia if elevation is rapid.¹⁰ In our experimental setting, elevation of [K⁺]₀ from 4 to 6 and 8 mM cardioverted AF in 33 and 100% of atria, respectively. Yet, elevation of [K⁺]₀ from 4 to 5 mM did not terminate AF (FIG. 2). Thus, it appears that mild elevation of [K⁺]₀ alone within its normal range encountered clinically has a low potential for AF cardioversion.

The efficacy of I_{Na} blockers (including flecainide) to inhibit the sodium channel is highly voltage-dependent, i.e., the more depolarized the RMP the greater the inhibition.^{3,11} It is well known also that elevation of [K⁺]₀ depolarizes the cardiac RMP.^{1,2} The effect of [K⁺]₀ elevation to enhance the ability of I_{Na} blockers to inhibit the sodium channel is demonstrated herein. (FIG. 9)

Elevation of [K⁺]₀ itself “blocks” the sodium channel by reducing the availability of the channel (secondary to a depolarization of atrial RMP).^{1,2} At a significantly elevated [K⁺]₀ (≥6 mM) this reduction can be sufficient to cardiovert AF (40% at 6 mM and 100% at 8 mM in our experimental settings) without administration of a sodium channel blocker. Under experimental conditions used herein, elevation of [K⁺]₀ from 4 to 5 mM did not terminate AF (FIG. 2).

Hypokalemia is very common in hospitalized patients (about 20%)¹² and hypokalemia is a risk factor for AF incidence.^{13,14} A reduced efficiency of flecainide to stop AF at 3 mM vs. 4 mM [K⁺]₀, as identified herein (FIG. 2), has an important clinical implication. These observations indicate that AF cardioversion with I_{Na} blockers in patients with hypokalemia is inefficient and that correction of plasma [K⁺]₀ is advisable prior to cardioversion with sodium channel blockers.

The data presented herein demonstrate a potent synergistic effect of elevated [K⁺]₀ and I_{Na} block for acute rapid cardioversion of experimental AF (FIGS. 2 and 3). The novel approach of combining K elevation with I_{Na} block may be particularly useful in patients with persistent AF. Persistent AF is resistant to currently available pharmacological therapies. I_{Na} blockers, generally effective for cardioversion of paroxysmal AF, lose their efficacy in patients with persistent AF. A major reason for this failure is that in patients with persistent AF atrial cells display a more negative or hyperpolarized resting membrane potential (by 2-5 mV)^{15,16} which reduces the ability of I_{Na} blockers to depress excitability, thus rendering these drugs less effective for termination of AF. Elevation of [K⁺]₀ shifts the resting membrane potential of atrial cells in a positive direction, thus enhancing the ability of I_{Na} blockers to inhibit I_{Na} and depress excitability. As a consequence, elevation of plasma [K⁺]₀ is expected to restore and/or improve the effectiveness of I_{Na} blockers to cardiovert persistent AF.

This novel approach may be especially useful in patients with structural heart disease, which comprises a major fraction of AF patients.⁵ A critical limitation of many FDA-approved anti-AF agents (including I_{Na} blockers, such as flecainide and propafenone) is the risk of induction of ventricular arrhythmias in patients with significant structural heart diseases.⁵ A mild elevation in serum [K⁺]₀ (safe by itself) reduces the concentration of I_{Na} blockers needed to cardiovert AF, thus decreasing the risk of ventricular proarrhythmia.

Another possible use of this novel method would improve the outcome of the “pill in the pocket” approach in termination of AF. The “pill-in-the-pocket” approach involves the administration of a class IC antiarrhythmic, e.g. flecainide or propafenone, following episodes of paroxysmal AF in patients who are able to sense the onset of AF because of palpitations.¹⁷ It is generally initiated by the cardiologist following extensive evaluation to rule out structural heart disease and other conduction abnormalities. This allows termination of the suspected episode of AF at home without having to present to an ED or clinic. The pre-administration or co-administration of potassium tablets with flecainide or propafenone would greatly improve the ability of the “pill in the pocket” approach to terminate AF.

One more possible use of this novel approach would improve the outcome of inhalation therapy to cardiovert AF. Flecainide delivered via oral inhalation has recently been shown to be efficacious in restoring sinus rhythm in approximately 50% of patients with recent-onset AF in a Phase 2, open-label trial INSTANT (INhalation of flecainide to convert recent-onset SympTomatic Atrial fibrillatioN to sinus rhy Thm).¹⁸ The pre-administration of potassium tablets prior to flecainide inhalation would greatly improve the conversion of AF to sinus rhythm in this proposed home therapy.

The risk of induction of ventricular proarrhythmia by anti-AF agents by anti-AF agents is a major limitation of the pharmacological approach to rhythm control. This risk can be reduced or eliminated by atrial-selective agents.^{4,19,20} As indicated herein, combined administration of flecainide and mild elevation of [K⁺]₀ produced clear atrial-selective depression of sodium-channel-mediated parameters (i.e., induction of PRR and increase in DTE), displaying a relatively low risk for induction of ventricular proarrhythmia with this novel approach.

In the clinic, enduring maintenance of sinus rhythm after AF cardioversion is desirable, although recurrence of AF is often encountered soon after cardioversion. In our experimental setting, AF burden after AF termination with elevated [K⁺]₀ and flecainide was low. That modest elevation of plasma potassium in combination with I_{Na} block after AF cardioversion reduces AF burden in the long term. The need for continued treatment is expected to decrease with prolonged maintenance of sinus rhythm due to progressive reversal of atrial electrical and structural remodeling.

Our experimental data indicates that a modest elevation of serum [K⁺]₀, as small as 1 mM, significantly enhances the ability of I_{Na} blockers to acutely cardiovert AF and prevent its recurrence in the short term. A combination of I_{Na} block with a moderate elevation of serum potassium (not exceeding 5.5 mM) may be a novel approach to effectively, rapidly, and safely cardiovert AF as well as prevent its recurrence in the short term. In light of the potent effect of the combination of I_{Na} block and elevation of [K⁺]₀ to cause rate-dependent depression of atrial excitability, a long-lasting use of this combination reduces AF burden over the long term.

### REFERENCES

1. Miyata A, Dowell J D, Zipes D P, Rubart M. Rate-dependent [K+](o) accumulation in canine right atria in vivo: electrophysiological consequences. Am J Physiol Heart Circ Physiol. 2002;283(2):H506-H517.
2. Buchanan J W, Jr., Saito T, Gettes L S. The effects of antiarrhythmic drugs, stimulation frequency, and potassium-induced resting membrane potential changes on conduction velocity and dV/dtmax in guinea pig myocardium. Circ Res. 1985;56(5):696-703.
3. Carmeliet E, Mubagwa K. Antiarrhythmic drugs and cardiac ion channels: mechanisms of action. ProgBiophysMolBiol. 1998; 70(1):1-72.
4. Burashnikov A, Di Diego J M, Zygmunt A C, Belardinelli L, Antzelevitch C. Atrium-selective sodium channel block as a strategy for suppression of atrial fibrillation: differences in sodium channel inactivation between atria and ventricles and the role of ranolazine. Circulation. 2007;116(13):1449-1457.
5. Fuster V, Ryden L E, Cannom D S, et al. 2011 ACCF/AHA/HRS Focused Updates Incorporated Into the ACC/AHA/ESC 2006 Guidelines for the Management of Patients With Atrial Fibrillation A Report of the American College of Cardiology Foundation/American Heart Association Task Force on Practice Guidelines Developed in partnership with the European Society of Cardiology and in collaboration with the European Heart Rhythm Association and the Heart Rhythm Society. J Am Coll Cardiol. 2011;57(11):e101-e198.
6. Pandit S V, Zlochiver S, Filgueiras-Rama D, et al. Targeting atrio-ventricular differences in ion channel properties for terminating acute atrial fibrillation in pigs. Cardiovasc Res. 2011; 89:843-851.
7. Romeo E, D'Alto M, Santarpia G, et al. Hyperkalemia-induced conversion of permanent atrial fibrillation to normal sinus rhythm: a case report. Journal of cardiovascular medicine. 2011;12(9):678-680.
8. Yoon J H, Jung D H, Park S K, et al. Spontaneous sinus conversion of permanent atrial fibrillation during treatment of hyperkalemia. Korean Circ J. 2012;42(1):65-68.
9. Mathew PK. Hyperkalemia-induced conversion of chronic atrial fibrillation to normal sinus rhythm; a case report. Angiology. 1979;30(2):143-146.
10. Tazmini K, Fraz M S A, Nymo S H, Stokke M K, Louch W E, Øie E. Potassium infusion increases the likelihood of conversion of recent-onset atrial fibrillation-A single-blinded, randomized clinical trial. Am Heart J. 2020;221:114-124.
11. Syeda F, Holmes A P, Yu T Y, et al. PITX2 Modulates Atrial Membrane Potential and the Antiarrhythmic Effects of Sodium-Channel Blockers. J Am Coll Cardiol. 2016;68(17):1881-1894.
12. Cohn J N, Kowey P R, Whelton P K, Prisant L M. New guidelines for potassium replacement in clinical practice: a contemporary review by the National Council on Potassium in Clinical Practice. Arch Intern Med. 2000;160(16):2429-2436.
13. Krijthe B P, Heeringa J, Kors J A, et al. Serum potassium levels and the risk of atrial fibrillation: the Rotterdam Study. Int J Cardiol. 2013;168(6):5411-5415.
14. Farah R, Nassar M, Aboraya B, Nseir W. Low serum potassium levels are associated with the risk of atrial fibrillation. Acta Cardiol. 2021;76(8):887-890.
15. Bosch R F, Zeng X, Grammer J B, Popovic K, Mewis C, Kuhlkamp V. Ionic mechanisms of electrical remodeling in human atrial fibrillation. CardiovascRes. 1999;44(1):121-131.
16. Wettwer E, Christ T, Endig S, et al. The new antiarrhythmic drug Vernakalant: ex-vivo study of human atrial tissue from sinus rhythm and chronic atrial fibrillation. Cardiovasc Res. 2013;98(1):145-154.
17. Alboni P, Botto G L, Baldi N, et al. Outpatient treatment of recent-onset atrial fibrillation with the “pill-in-the-pocket” approach. NEnglJ Med. 2004;351(23):2384-2391.
18. Crijns H, Elvan A, Al-Windy N, et al. Open-Label, Multicenter Study of Flecainide Acetate Oral Inhalation Solution for Acute Conversion of Recent-Onset, Symptomatic Atrial Fibrillation to Sinus Rhythm. Circ Arrhythm Electrophysiol. 2022;15(3):e010204.
19. Nattel S, Carlsson L. Innovative approaches to anti-arrhythmic drug therapy. NatRevDrug Discov. 2006;5(12):1034-1049.
20. Burashnikov A, Antzelevitch C. New development in atrial antiarrhythmic drug therapy. Nat RevCardiol. 2010;7(3):139-148.

Each and every patent, patent application, and publication, including publications herein and publicly available nucleic acid and amino acid sequences cited throughout the disclosure, is expressly incorporated herein by reference in its entirety. The contents of US Provisional Ser. No. 63/371,601 is incorporated herein this invention has been disclosed with reference to specific embodiments, it is apparent that other embodiments and variations of this invention are devised by others skilled in the art without departing from the true spirit and scope of the invention. The appended claims include such embodiments and equivalent variations.

What is claimed is:

1. A method of treating a cardiac arrhythmia, the method comprising administering a sodium channel blocker to a patient having elevated extracellular potassium levels.

2. A method of treating a cardiac arrhythmia, the method comprising,
a. administering an agent that elevates extracellular potassium levels and
b. administering a sodium channel blocker.

3. The method of claim 2, wherein the agent that elevates extracellular potassium levels is administered prior to administering the sodium channel blocker.

4. The method of claim 2, wherein the agent that elevates extracellular potassium levels is administered at substantially the same time as the sodium channel blocker.

5. The method of any one of claims 2-4, wherein the agent is an oral potassium supplement or intravenous potassium chloride.

6. The method of any one of claims 1 to 5, wherein the agent that elevates extracellular potassium levels and/or the sodium channel blocker is in a solid or liquid formulation.

7. The method of any one of the preceding claims, wherein termination of AF is increased by at least 20%, at least 30%, at least 40%, at least 50%, at least 60%, at least 70%, at least 80%, at least 90%, or at least 100%, when compared to a patient with an extracellular potassium level of 4 mM or less.

8. The method of any one of the preceding claims, wherein duration of AF is decreased by at least 20%, at least 30%, at least 40%, at least 50%, at least 60%, at least 70%, at least 80%, at least 90%, or at least 100% when compared to a patient with an extracellular potassium level of 4 mM or less.

9. The method of any one of the preceding claims, wherein said method prolongs AF cycle length.

10. The method of claim 7, wherein the AF cycle length is prolonged when compared to a patient with baseline potassium levels.

11. The method of any of the preceding claims, wherein risk of ventricular proarrhythmia is decreased.

12. The method of any one of the preceding claims, wherein the cardiac arrhythmia is a supraventricular tachycardia.

13. The method of any one of the preceding claims, wherein the cardiac arrhythmia is atrial fibrillation (AF).

14. The method of any one of the preceding claims, wherein the cardiac arrhythmia is persistent or sustained AF.

15. The method of any one of the preceding claims, wherein the patient has a structural heart disease.

16. The method of any one of the preceding claims, wherein the cardiac arrythmia is resistant to treatment with a sodium channel blocker alone.

17. The method of any one of the preceding claims, wherein the elevated potassium level is greater than 4 mM.

18. The method of anyone of the preceding claims, wherein the elevated potassium level is greater than or equal to 5 mM.

19. The method of any one of the preceding claims, wherein the elevated potassium level is between 5 mM and 5.5 mM.

20. The method of any one of the preceding claims, wherein the elevated potassium level is about 5.5 mM.

21. The method of any one of the preceding claims, wherein baseline potassium level is between 2.0 and 4.5 mM.

22. The method of any one of the preceding claims, wherein baseline potassium level is between 2.5 and 4.5 mM.

23. The method of any one of the preceding claims, wherein baseline potassium level is between 3.0 and 4.0 mM.

24. The method of any one of the preceding claims, wherein the elevated potassium level at least 0.5 mM greater than the base line potassium level.

25. The method of any one of the preceding claims, wherein the elevated potassium levels is between 0.5 mM-0.8 mM greater than the base line potassium level.

26. The method of any one of the preceding claims, wherein the elevated potassium levels is at least 1.0 mM greater than the base line potassium levels.

27. The method of any one of the preceding claims, wherein the sodium channel blocker is a class IC sodium channel blocker.

28. The method of any one of the preceding claims, wherein the sodium channel blocker is a class IB sodium channel blocker.

29. The method of any one of the preceding claims, wherein the sodium channel blocker is a class IA sodium channel blocker.

30. The method of any one of the preceding claims, wherein the sodium channel blocker is Flecainide or Propafenone.

31. The method of any one of the preceding claims, wherein the sodium channel blocker is administered by oral inhalation.

32. A method of treating cardiac arrhythmia, the method comprising administration of an agent that elevates extracellular potassium levels to at least 4 mM.

33. A kit for performing any of the preceding methods, the kit comprising a sodium channel blocker and an agent that elevates extracellular potassium levels.

34. A method of treating cardiac arrhythmia, the method comprising administration of an agent that elevates extracellular potassium levels by at least 0.5 mM over baseline extracellular potassium levels.

35. An oral dosage composition comprising a sodium channel blocker and an agent that elevates extracellular potassium levels.

36. The composition of claim 35, wherein the sodium channel blocker is Flecainide or Propafenone.

37. A method of treating cardiac arrhythmia in a patient in need thereof, the method comprising,
i) obtaining a biological sample from said patient,
ii) measuring baseline extracellular potassium levels in said biological sample,
iii) administering an agent that elevates extracellular potassium levels at least 0.5 mM over baseline extracellular potassium levels and
iv) administering a sodium channel blocker.
